(19) United States
(12) Patent Application Publication
(10) Pub. No.: US 2018/0247717 A1
(43) Pub. Date: Aug. 30, 2018

(54) SYSTEM AND METHOD FOR FACILITATING A PROMOTIONAL EVENT

(71) Applicant: HSC ACQUISITION, LLC, Mahwah, NJ (US)

(72) Inventor: Ala Mahafzah, Wayne, NJ (US)

(21) Appl. No.: 15/852,812

(22) Filed: Dec. 22, 2017

Related U.S. Application Data
(63) Continuation of application No. 13/624,413, filed on Sep. 21, 2012, now abandoned.

(51) Int. Cl. G16H 80/00; G06Q 30/02

(52) U.S. Cl. CPC G16H 80/00; G06Q 30/0255

(57) ABSTRACT

In a method for facilitating a healthcare promotional event, a computer transmits data representative of an invitation for a healthcare professional, selected from a plurality of healthcare professionals, to participate in a healthcare promotional event, selected from a plurality of predefined healthcare promotional events, wherein participating in a healthcare promotional event comprises watching a pre-recorded healthcare promotional video and participating in a live communication with a healthcare promotional event host; receives confirmation of the healthcare professional's intent to participate in the healthcare promotional event; launches the healthcare promotional event by initiating playback of the pre-recorded healthcare promotional video responsive to the computer receiving notification that the healthcare professional has received a communication comprising pre-defined information associated with the pre-recorded healthcare promotional video; and establishes live communication with the healthcare promotional event host responsive to the computer receiving notification that the healthcare professional has completed watching the pre-recorded healthcare promotional video.

### CROSS-REFERENCE TO RELATED APPLICATIONS

This application is a continuation of U.S. patent application Ser. No. 13/624,413, filed Sep. 21, 2012, and entitled “SYSTEM AND METHOD FOR FACILITATING A PROMOTIONAL EVENT,” the entire contents of which are incorporated herein by reference.

### FIELD OF INVENTION

The present disclosure relates to the field of promotional events. More particularly, the present invention relates to a system and method for facilitating healthcare promotional events.

### BACKGROUND

Industry opinion leaders are individuals considered to be effective at influencing others within an industry to make certain choices and to conform their opinions to be consistent with those of the opinion leader. In the healthcare industry specifically, a key opinion leader (KOL) is a physician who is capable of influencing other physicians to prescribe a certain drug produced by a specific drug manufacturer. Thus, it is desirable for a healthcare marketing company that is promoting a specific drug, to arrange a promotional event in which a KOL is able to share his opinion regarding the drug with other healthcare professionals.

One type of healthcare promotional event is a dinner meeting of physicians, featuring a KOL speaking about a particular drug. Promotional events requiring such physical presence, however, may not be practical for some physicians due to time constraints and geographic constraints. Moreover, organizing a healthcare promotional event requires adherence to certain industry regulations and compliance standards, which may be burdensome for a healthcare marketing company to document and comply with.

### SUMMARY OF THE INVENTION

A method for facilitating a healthcare promotional event comprises the step of a computer transmitting data representative of an invitation for a healthcare professional, selected from a plurality of healthcare professionals, to participate in a healthcare promotional event, selected from a plurality of predefined healthcare promotional events, wherein participating in a healthcare promotional event comprises watching a pre-recorded healthcare promotional video and participating in a live communication with a healthcare promotional event host. The method further comprises the step of a computer receiving confirmation of the healthcare professional's intent to participate in the healthcare promotional event. The method further comprises the step of a computer launching the healthcare promotional event by initiating playback of the pre-recorded healthcare promotional video responsive to the computer receiving notification that the healthcare professional has received a communication comprising pre-defined information associated with the pre-recorded healthcare promotional video. The method further comprises the step of a computer establishing live communication with the healthcare promotional event host responsive to the computer receiving notification that the healthcare professional has completed watching the pre-recorded healthcare promotional video.

A system for facilitating a healthcare promotional event comprises a hub computer configured to host a live video conference with a healthcare key opinion leader associated with a healthcare promotional event. The system also comprises a spoke computer including at least one processor, at least one computer-readable tangible storage device, and program instructions stored on the at least one storage device for execution by the at least one processor. The program instructions inlcude first program instructions configured to associate a healthcare professional with the healthcare promotional event. The program instructions further include second program instructions configured to initiate playback of a healthcare promotional video associated with the healthcare promotional event. The program instructions further include third program instructions configured to establish a connection, via the hub computer, to the live video conference with the healthcare key opinion leader. The program instructions further include fourth program instructions configured to certify that the healthcare professional associated with the healthcare promotion event participated in the healthcare promotional event.

A computer program product for facilitating a virtual promotional event comprising at least one computer-readable tangible storage device and program instructions stored on the at least one storage device. The program instructions include first program instructions configured to transmit data representative of an invitation for an invitee, selected from a predefined dataset, to participate in a virtual promotional event, selected from a plurality of predefined virtual promotional events, wherein participating in a virtual promotional event comprises watching a pre-recorded promotional video and participating in a live communication with a virtual promotional event host. The program instructions further include second program instructions configured to receive confirmation of the invitee's intent to participate in the virtual promotional event and to change a status of an invitee from an invited status to a confirmed status, responsive to receiving the confirmation. The program instructions further comprise third program instructions configured to receive notification that the confirmed invitee has received a communication comprising pre-defined information associated with the promotional video, and in response, to launch the virtual promotional event by initiating playback of the pre-recorded promotional video. The program instructions further include fourth program instructions configured to receive notification that the confirmed invitee has completed watching the pre-recorded promotional video, and in response, to establish communication with the virtual promotional event host.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for facilitating a promotional event.

FIG. 2 illustrates a block diagram of an example system for facilitating a promotional event.

FIG. 3 is a flow chart illustrating an example method for facilitating a promotional event.

FIG. 4 is a schematic diagram of an example computer for implementing the example system of FIG. 1 for facilitating a promotional event.

### DETAILED DESCRIPTION

The following includes definitions of selected terms employed herein. The definitions include various examples, forms, or both of components that fall within the scope of a term and that may be used for implementation. The examples are not intended to be limiting. Both singular and plural forms of terms may be within the definitions.

“Computer communication,” as used herein, refers to a communication between two or more computing devices (e.g., computer, personal digital assistant, cellular telephone) and can be, for example, a network transfer, a file transfer, an applet transfer, an email, a hypertext transfer protocol (HTTP) transfer, and so on. A computer communication can occur across, for example, a wireless system (e.g., IEEE 802.11, IEEE 802.15), an Ethernet system (e.g., IEEE 802.3), a token ring system (e.g., IEEE 802.5), a local area network (LAN), a wide area network (WAN), a point-to-point system, a circuit switching system, a packet switching system, combinations thereof, and so on.

“Computer-readable medium,” as used herein, refers to a medium that participates in directly or indirectly providing signals, instructions, or data. A computer-readable medium may take forms, including, but not limited to, non-volatile media, volatile media, and transmission media. Non-volatile media may include, for example, optical or magnetic disks, and so on. Volatile media may include, for example, optical or magnetic disks, dynamic memory, and the like. Transmission media may include coaxial cables, copper wire, fiber optic cables, and the like. Transmission media can also take the form of electromagnetic radiation, like that generated during radio-wave and infra-red data communications, or take the form of one or more groups of signals. Common forms of a computer-readable medium include, but are not limited to, a floppy disk, a flexible disk, a hard disk, a magnetic tape, other magnetic media, a CD-ROM, other optical media, punch cards, paper tape, other physical media with patterns of holes, a RAM, a ROM, an EPROM, a FLASH-EPROM, or other memory chip or card, a memory stick, a carrier wave/pulse, Phase Change Memory, and other media from which a computer, a processor, or other electronic device can read. Signals used to propagate instructions or other software over a network, like the Internet, can be considered a “computer-readable medium.”

“Data store,” as used herein, refers to a physical or logical entity that can store data. A data store may be, for example, a database, a table, a file, a list, a queue, a heap, a memory, a register, and so on. A data store may reside in one logical or physical entity or may be distributed between two or more logical or physical entities.

“Logic,” as used herein, includes but is not limited to hardware, firmware, software, or combinations of each to perform a function(s) or an action(s), or to cause a function or action from another logic, method, or system. For example, based on a desired application or needs, logic may include a software controlled microprocessor, discrete logic like an application specific integrated circuit (ASIC), a programmed logic device, a memory device containing instructions, or the like. Logic may include one or more gates, combinations of gates, or other circuit components. Logic may also be fully embodied as software. Where multiple logical logics are described, it may be possible to incorporate the multiple logical logics into one physical logic. Similarly, where a single logical logic is described, it may be possible to distribute that single logical logic between multiple physical logics.

An “operable connection,” or a connection by which entities are “operably connected,” is one in which signals, physical communications, or logical communications may be sent or received. Typically, an operable connection includes a physical interface, an electrical interface, or a data interface, but it is to be noted that an operable connection may include differing combinations of these or other types of connections sufficient to allow operable control. For example, two entities can be operably connected by being able to communicate signals to each other directly or through one or more intermediate entities like a processor, operating system, a logic, software, or other entity. Logical or physical communication channels can be used to create an operable connection.

“Software,” as used herein, includes but is not limited to, one or more computer or processor instructions that can be read, interpreted, compiled, or executed and that cause a computer, processor, or other electronic device to perform functions, actions, or behave in a desired manner. The instructions may be embodied in various forms like routines, algorithms, modules, methods, threads, or programs including separate applications or code from dynamically or statically linked libraries. Software may also be implemented in a variety of executable or loadable forms including, but not limited to, a stand-alone program, a function call (local or remote), a servelet, an applet, instructions stored in a memory, part of an operating system, or other types of executable instructions. It will be appreciated by one of ordinary skill in the art that the form of software may depend, for example, on requirements of a desired application, the environment in which it runs, or the desires of a designer/programmer or the like. It will also be appreciated that computer-readable or executable instructions can be located in one logic or distributed between two or more communicating, co-operating, or parallel processing logics and thus can be loaded or executed in serial, parallel, massively parallel, and other manners.

Suitable software for implementing the various components of the example systems and methods described herein may be produced using programming languages and tools like Java, Java Script, Java.NET, ASP.NET, VB.NET, Cocoa, Pascal, C#, C++, C, CGI, Perl, SQL, APIs, SDKs, assembly, firmware, microcode, or other languages and tools. Software, whether an entire system or a component of a system, may be embodied as an article of manufacture and maintained or provided as part of a computer-readable medium as defined previously. Another form of the software may include signals that transmit program code of the software to a recipient over a network or other communication medium. Thus, in one example, a computer-readable medium has a form of signals that represent the software/firmware as it is downloaded from a web server to a user. In another example, the computer-readable medium has a form of the software/firmware as it is maintained on the web server. Other forms may also be used.

“User,” as used herein, includes but is not limited to one or more persons, software, computers or other devices, or combinations of these.

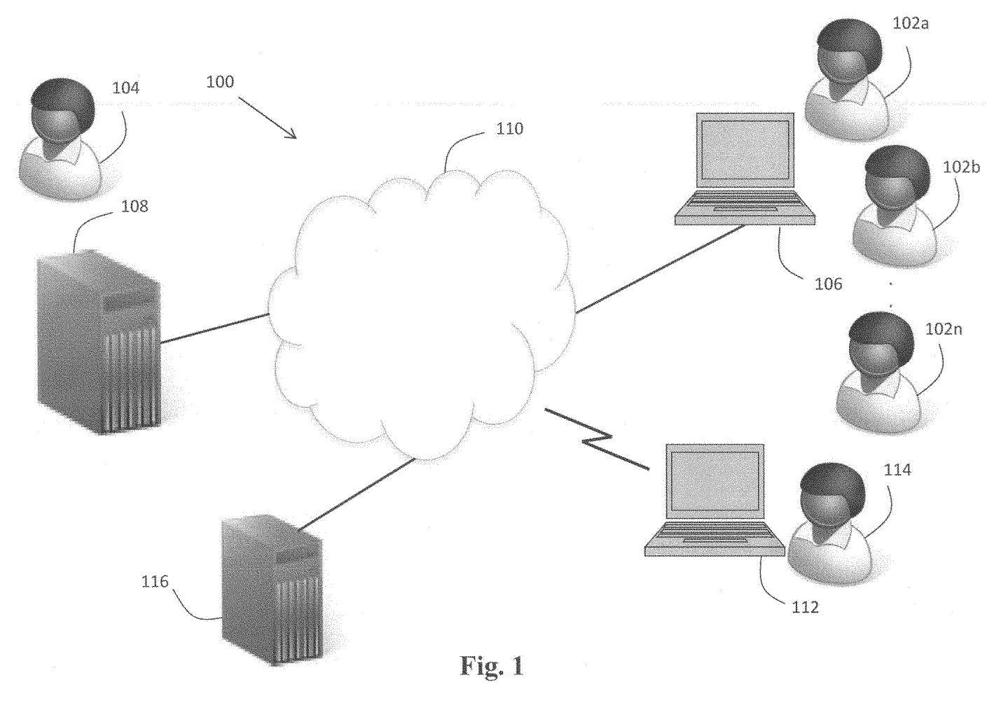

FIG. 1 illustrates an example system 100 for facilitating a promotional event. It should be understood that, although system 100 is described in the context of facilitating a healthcare promotional event, system 100 may also be used to facilitate promotional events in other field for a variety of applicable products or services as deemed appropriate by an event organizer. System 100 enables an event organizer to connect one or more healthcare professionals 102a-102n (hereinafter referred to as healthcare professional 102) to an event host 104.

Event host 104 is a healthcare subject matter expert, or a healthcare key opinion leader (hereinafter referred to as “KOL”), that is qualified to discuss with healthcare professional 102, a drug or other product being promoted and to answer questions about the drug. System 100 enables a virtual, real time, question and answer session (hereinafter referred to as “Q/A session”) between event host 104 and healthcare professional 102, regarding the drug. Reference to a virtual session is meant to imply that healthcare professional 102 is able to communicate and interact with event host 104 without being physically present in the same location as event host 104. Prior to connecting healthcare professional 102 with event host 104, the system 100 displays a prerecorded video about the drug to the healthcare professional 102 to educate and provide healthcare professional 102 with background information about the drug.

Thus, in one example embodiment, a promotional event includes two components: healthcare professional 102 is shown a prerecorded video, after which healthcare professional 102 is virtually connected with event host 104 for a real time Q/A session. System 100 enables an event organizer to facilitate such a virtual promotional event. It should be understood that a promotional event may include additional components which system 100 may be configured to facilitate as well. For example, an event organizer may wish to include in a promotional event a survey or other similar types or interactive components.

System 100 includes a spoke computer 106 which is configured to enable healthcare professional 102 to watch the pre-recorded video and to communicate with event host 104 in real time. It should be understood that, although spoke computer 106 is depicted as laptop computer, spoke computer 106 may also be a tablet computer, a mobile phone, or any other type of suitable mobile computing device. Spoke computer 106 may include a display or it may project onto a television or other type of display screen. System 100 also includes a hub computer 108 which is configured to enable event host 104 connect with healthcare professional 102 in order to participate in the live Q/A session portion of the promotional event. It should be understood that, although hub computer 108 is depicted as a computer server or a mainframe, hub computer can by any suitable computing device such as a laptop computer, a desktop computer, a tablet computer, a mobile phone, and so on. Spoke computer 106 and hub computer 108 are configured to communicate via Internet 110, either wirelessly or using a wired connection. It should be understood that spoke computer 106 and hub computer 108 may also be configured to communicate via other suitable means such a local area network (LAN) or a wide area network (WAN).

In an example embodiment, system 100 also includes a second spoke computer 112 for enabling a second healthcare professional 114 to participate in the same promotional event, even though second healthcare professional 114 may be physically located in a different geographic location as compared to healthcare professional 102. For example, healthcare professional 102 may be at a first location while second healthcare professional 114 may be at a second location. A first event organizer and a second event organizer, physically present at first location and at second location, each provide healthcare professional 102 and second healthcare professional 114, respectively, with spoke computer 106 and second spoke computer 112 to enable them to watch the same prerecorded video. Spoke computer 106 and second spoke computer 112 are both configured to establish a connection with hub computer 108, via Internet 110, at a scheduled time in order for healthcare professional 102 and second healthcare professional 114 to participate in the live Q/A session with event host 104.

It should be understood that, although system 100 is illustrated to include two spoke computers, system 100 may include three or more spoke computers to facilitate participation in a virtual promotional event by healthcare professionals in three or more physical locations. Thus, system 100 implements a hub and spoke model in which multiple healthcare professionals located in multiple locations can connect, via a spoke computer, to an event host at a hub computer, to participate in a live communication session with the event host. The healthcare professionals located at, and participating through, a spoke computer correspond to spoke events while the event host participating via the hub computer represents a hub event.

In one example embodiment, system 100 includes a storage computer 116 which is configured to store information about healthcare professionals, information about predefined healthcare promotional events, and prerecorded healthcare promotional videos. Thus, in one example embodiment, spoke computer 106 and second spoke computer 112 are configured to synchronize data with storage computer 116, to help facilitate scheduling spoke promotional events. It should be understood that, although storage computer 116 is depicted as a computer server or a mainframe, storage computer 116 may be a desktop computer, a laptop computer, or any other suitable computing device capable of storing data.

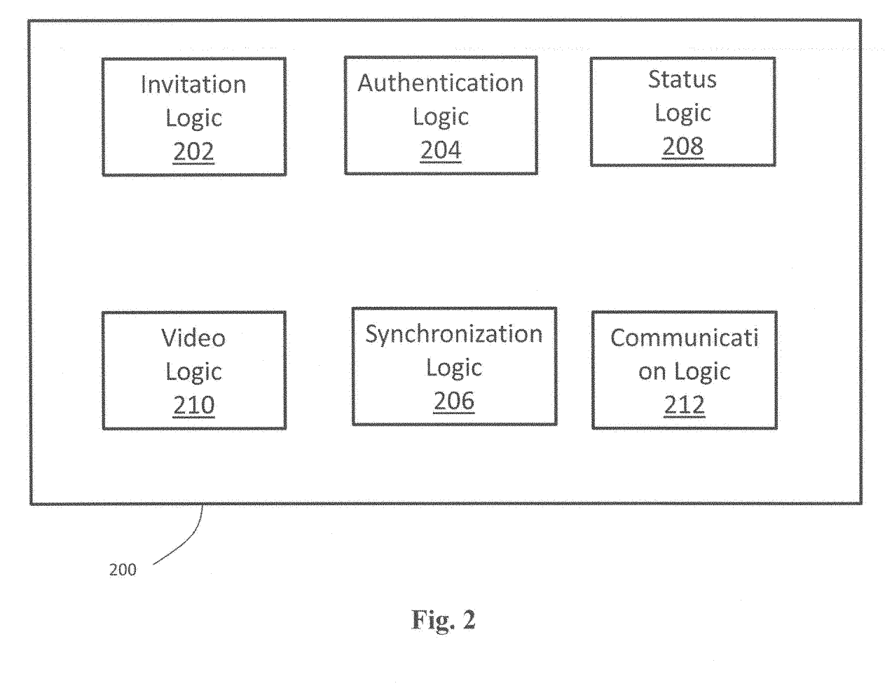

A spoke computer includes a system for managing spoke promotional events for facilitating participation in spoke promotional events. FIG. 2 illustrates a block diagram of an example system 200 for facilitating a promotional event, as implemented by a spoke computer. System 200 has an invitation logic 202 configured to enable an event organizer to select a promotional event from one or more predefined promotional events, and to select one or more healthcare professionals from one or more predefined healthcare professionals to invite to the promotional event. In other words, the event organizer associates certain healthcare professionals with a predefined promotional event. This association creates a spoke event. Invitation logic 202 enables an event organizer to create multiple spoke events corresponding to multiple hub events. Invitation logic 202 also enables an event organizer to view information about and manage previously created spoke events. For example, an event organizer may load a previously created spoke even and view information, such as a resume, about the KOL scheduled to participate in the Q/A session. An event organizer may also manage the invitee list by removing already invited guests or by inviting additional guests. An even organizer may also cancel a spoke event.

A promotional event is predefined by promotional event data which includes information necessary for a healthcare professional to connect with and participate in the promotional event. For example, a predefined promotional event includes information about what drug or product is going to be promoted, which prerecorded video a participant is required to watch before participating in the live Q/A session, the name and other relevant information about the subject matter expert or KOL that is going to be hosting the live Q/A session, the date and time of the live Q/A session, specifications for how to connect with the live Q/A session, and so on. Promotional events are defined or created by a systems administrator in advance using a systems administration tool. Specifications for how to connect with a live Q/A session may include, for example, an Adobe Connect URL for connecting with an Adobe Connect session, a WebEx or a GoToMeting link, or a phone number for calling into a teleconference.

Invitation logic 202 is further configured to transmit an invitation to the one or more healthcare professionals, selected by the event organizer, to attend the virtual promotional event. In an example embodiment, invitation logic 202 is configured to send an electronic mail invitation. In an another example embodiment, invitation logic 202 is configured to send a short message service (SMS) message or other suitable electronic message in order to invite a healthcare professional to attend a virtual promotional event. Invitation logic 202 is also configured to transmit an electronic communication, such as an electronic mail or an SMS message, to a previously invited healthcare professional to inform the healthcare professional that a promotional event was canceled. Invitation logic 202 is further configured to receive, from an event organizer, a reason for canceling the spoke event, such as “Insufficient RSVPS,” or “Connection Issue,” and transmit the reason to the healthcare professional when transmitting the cancelation notice.

In an example embodiment, system 200 includes an authentication logic 204 configured to receive authentication credentials from an event organizer. Based on the received credentials, authentication logic 204 is configured to filter the predefined promotional events and healthcare professionals made available to the event organizer. For example, an event organizer may limited to selecting those healthcare professionals residing in a specific geographical region assigned to the event organizer. Similarly, the event organizer may be limited to selecting promotional events related to specific drugs or products which the event organizer has been assigned to promote.

In one example embodiment, system 200 includes a synchronization logic 206 to synchronize data on spoke computer 106 with data on storage computer 116. Specifically, synchronization logic 206 is configured to synchronize information about healthcare professionals, information about predefined promotional events, and pre-recorded promotional videos. Thus, if a systems administrator creates a new promotional event and stores the event data on storage computer 116, synchronization logic 206 is configured to retrieve information about the new promotional event in order to enable an event organizer to create an associated spoke event, at spoke computer 106. In one example, synchronization logic 206 may be configured to synchronize data based on the authentication of an event organizer. For example, synchronization logic 206 may be configured to synchronize a spoke computer with spoke events, or associations between healthcare professionals and a predefined promotional event, that an event organizer previously defined on another spoke computer. Thus, an event organizer is not limited to using the same spoke computer.

System 200 also includes a status logic 208 configured to receive confirmation that a healthcare professional intends to participate in the healthcare promotional event to which the healthcare professional was invited. Status logic 208 is also configured to receive confirmation that a healthcare professional does not intend to participate in the healthcare promotional event to which the healthcare professional was invited. In one example embodiment, status logic 208 receives the confirmation via manual input by the event organizer. For example, the event organizer may receive confirmation from a healthcare professional, via SMS text message, email, phone call, or other suitable means and then manually provide the confirmation to status logic 208 via a user input device (not shown). In another example embodiment, status logic 208 may receive a confirmation directly from a healthcare professional. For example, status logic 208 may be configured to receive an SMS text message, an email, or another suitable electronic communication, and process the electronic communication in order to determine whether the healthcare professional intends to participate in the promotional event.

Status logic 208 is also configured to change a status of an invited healthcare professional with respect to a specific promotional event. For example, status logic 208 is configured to change the status of a healthcare professional, with respect to a specific promotional event, from an “invited” status to a “confirmed” status, or other similar status. Similarly, status logic 208 is also configured to change the status of a healthcare professional, with respect to a specific promotional event, from an “invited” status to a “declined” status, or other similar status. Status logic 208 is configured to store status data in a database, either locally at spoke computer 106, or to synchronize the status data with storage computer 116.

System 200 also includes a video logic 210 configured to initiate playback of a pre-recorded healthcare promotional video. In an example embodiment, a pre-recorded healthcare promotional video is locally stored on spoke computer 106. In another example embodiment, a pre-recorded healthcare promotional video is streamed, via Internet 110, to spoke computer 106 during playback.

In an example embodiment, a healthcare professional may be required to read specific Important Safety Information (ISI) or other related documentation before being allowed to watch the pre-recorded healthcare promotional video and to participate in the promotional event. Thus, in one example embodiment, video logic 210 is configured to communicate to the healthcare professional predefined information such as ISI associated with the pre-recorded healthcare promotional video and the drug being promoted. In this example, video logic 210 is configured to initiate playback of the prerecorded healthcare promotional video only after receiving confirmation from an event organizer that the healthcare professional has read the ISI. For example, an event organizer may be asked to click a button in order to confirm that the healthcare professional has read the ISI. In one example embodiment, video logic 210 is configured to display the ISI at any point during the promotional event if a healthcare professional desires to refer back to the ISI.

It should be understood that, although video logic 210 is described within the context of system 200 for facilitating a promotional event, video logic 210 may also be configured to initiate playback of pre-recorded videos independently of a promotional event associated with a live Q/A session. For example, a sales representative may be in a sales meeting, discussing a drug or other product with a healthcare professional and may wish to play for the healthcare professional a particular video from a video library stored on spoke computer 106. However, there may not be a promotional event scheduled for that specific product at the time of the sales representative's sales visit. Thus, in one example, video logic 210 is configured to receive a selection for an on-demand video presentation and to initiate playback of the selected pre-recorded video without having to define a specific spoke event.

System 200 further includes a communication logic 212 configured to establish a communication with a healthcare promotional event host, such as a KOL. Accordingly, communication logic 212 enables a healthcare professional to communicate in real time with the KOL in order to ask questions and to further discuss and learn more about the product or drug being promoted. The live communication can be a video conference, a teleconference, or other suitable means for communicating in real time. For example, communication logic 212 may be configured to establish communication with a KOL using Adobe Connect or other suitable communication software. In such an example, a predefined promotional event may include specifications and access credentials for connecting with the KOL via Adobe Connect.

In an example embodiment, a healthcare professional may not be permitted to participate in a live Q/A session or other type of live communication with the KOL unless the healthcare professional has first successfully completed watching the corresponding pre-recorded video. Accordingly, in such an embodiment, communication logic 212 is configured to establish the communication with the KOL only after receiving confirmation that the healthcare professional has completed watching a corresponding pre-recorded video. In one example, video logic 210 is configured to receive such a confirmation from an event organizer and to provide the confirmation to communication logic 212. For example, an event organizer may be asked to click a button to confirm that the healthcare professional has completed watching the pre-recorded video.

In an example embodiment, industry regulations and compliance standards may require event organizers to track and report attendance at promotional events. Thus, in one example, status logic 208 is configured to not only confirm a healthcare professional's intent to attend a promotional event, but also to confirm that the healthcare professional did actually attend and participate in the promotional event. Specifically, status logic 208 is configured to receive confirmation from an event organizer that a healthcare professional participated in the promotional event. Status logic 208 is also configured to change a status of a healthcare professional from “confirmed” to “attended,” or other similar status, if an event organizer confirms that a healthcare professional did attend the promotional event. Similarly, status logic 208 is configured to change a status of a healthcare professional from “confirmed” to “no-show,” or other similar status, if an event organizer confirms that a healthcare professional did not attend the promotional event. Status logic 208 is configured to store attendance information in a database for future reporting and certification purposes.

When a promotional event is finished, before finalizing the list of attendees and closing out a promotional event, an event organizer may wish to manage the list of attendees to account for healthcare professionals that weren't originally invited to the attend the promotional event that nevertheless did attend the promotional event. Thus, status logic 208 is further configured to receive a notification that a non-invited healthcare professional did attend the promotional event and to store the corresponding data. Specifically, status logic 208 is configured associate a healthcare professional with the promotional event by adding him to the spoke event created by the event manager and to change the status of the healthcare professional to “attended,” or a similar status. Status logic 208 is also configured to store information about the non-invited healthcare professional if the non-invited healthcare professional is not a previously defined healthcare professional from which the original invitees were selected.

In an example embodiment, status logic 208 is configured to receive and store responses to various certification questions after a promotional event is complete in order to comply with industry compliance standards and regulations. For example, an event organizer may be asked to certify that the final healthcare professional attendee list is complete and accurate.

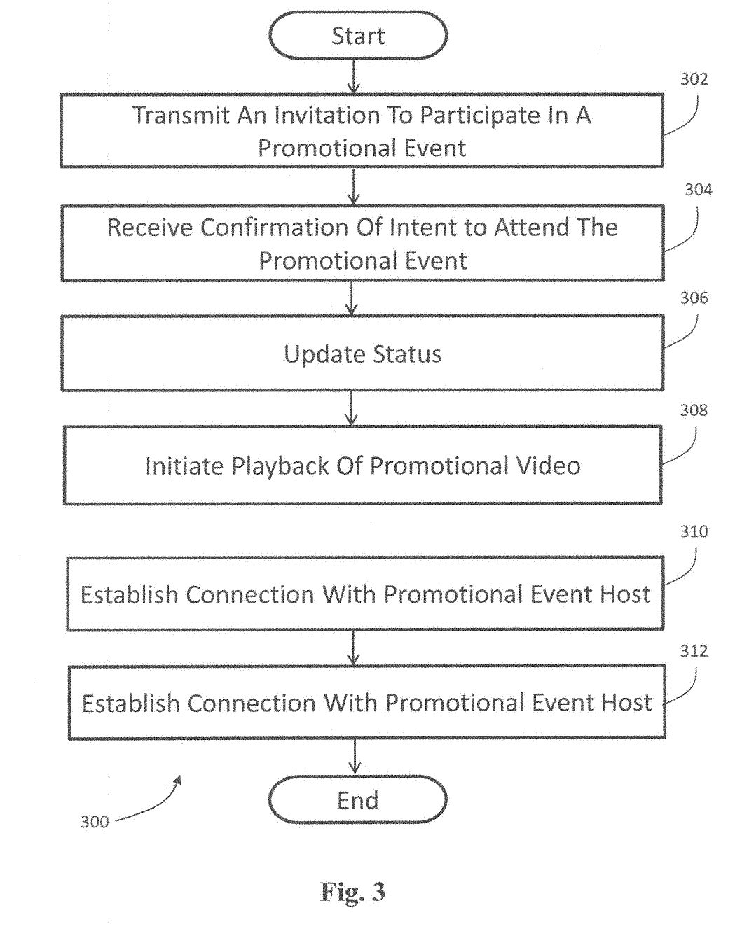

FIG. 3 is a flow chart illustrating an example method for facilitating a promotional event. At step 302, invitation logic 202 transmits an invitation in the form of an electronic message for a healthcare professional, selected from a predefined group of healthcare professionals, to participate in a promotional event, selected from a group of predefined healthcare promotional events. A healthcare promotional event includes a pre-recorded healthcare promotional video and a live communication with a promotional event host such as a KOL. In one example, invitation logic 202 transmits invitations to multiple healthcare professionals. It should be understood that, although the example method is described with respect to facilitating a healthcare promotional event, the example method can be implemented within any suitable industry, other than healthcare.

In an example embodiment, prior to transmitting the invitation, invitation logic 202 receives authentication credentials from an event organizer and filters the predefined healthcare professionals and the predefined promotional events from which the event organizer is able to select from, based on the received credentials.

At step 304, status logic 208 receives confirmation of the invited healthcare professional's intent to participate in the healthcare promotional event. In an example embodiment, at step 306, status logic 208 updates an invited healthcare professional's status, indicating intention to attend, responsive to receiving the confirmation.

At step 308, video logic 210 initiates playback of a pre-recorded healthcare promotional video. In an example embodiment, video logic 210 initiates playback of the promotional video responsive to receiving notification that the invited healthcare professional has received a communication including predefined information associated with the promotional video, such as Important Safety Information.

At step 310, communication logic 212 establishes a communication, such as a live video conference, with a healthcare promotional event host. In an example embodiment, communication logic 212 establishes the communication responsive to receiving notification that the healthcare professional has completed watching the promotional video.

In an example embodiment, at step 312, status logic 208 receives a notification indicating whether the invited healthcare professional participated in the promotional event and stores the information. In an example embodiment, status logic 208 receives a notification indicating that a non-invited healthcare professional participated in the promotional event. Accordingly, status logic 208 stores information to indicate that the non-invited healthcare professional participated in the promotional event and also stores information about the healthcare professional if the healthcare professional is not a predefined healthcare professional.

While for purposes of simplicity of explanation, the illustrated methodologies are shown and described as a series of blocks, it is to be appreciated that the methodologies are not limited by the order of the blocks, as some blocks can occur in different orders or concurrently with other blocks from that shown or described. Moreover, less than all the illustrated blocks may be required to implement an example methodology. Furthermore, additional or alternative methodologies can employ additional, not illustrated blocks.

In the flow diagram, blocks denote “processing blocks” that may be implemented with logic. The processing blocks may represent a method step or an apparatus element for performing the method step. A flow diagram does not depict syntax for any particular programming language, methodology, or style (e.g., procedural, object-oriented). Rather, a flow diagram illustrates functional information one skilled in the art may employ to develop logic to perform the illustrated processing. It will be appreciated that in some examples, program elements like temporary variables, routine loops, and so on, are not shown. It will be further appreciated that electronic and software applications may involve dynamic and flexible processes so that the illustrated blocks can be performed in other sequences that are different from those shown or that blocks may be combined or separated into multiple components. It will be appreciated that the processes may be implemented using various programming approaches like machine language, procedural, object oriented or artificial intelligence techniques.

In one example, methodologies are implemented as processor executable instructions or operations provided on a computer-readable medium. While the above method is described as being provided on a computer-readable medium, it is to be appreciated that other example methods described herein can also be provided on a computer-readable medium.

While FIG. 3 illustrate various actions occurring in serial, it is to be appreciated that various actions illustrated in FIG. 3 could occur substantially in parallel. While four processes are described, it is to be appreciated that a greater or lesser number of processes could be employed and that lightweight processes, regular processes, threads, and other approaches could be employed. It is to be appreciated that other example methods may, in some cases, also include actions that occur substantially in parallel.

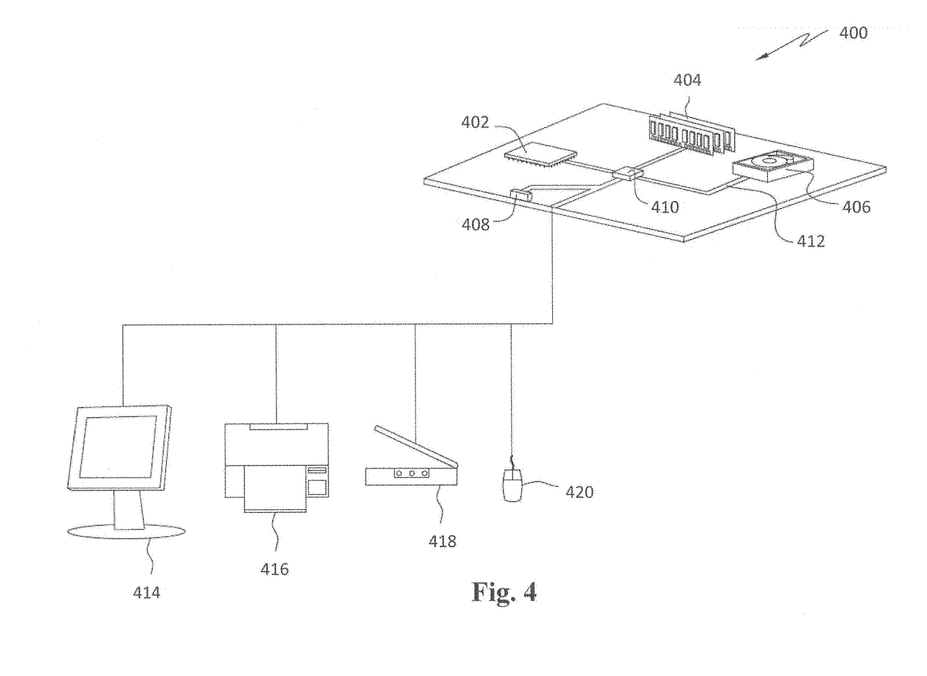

FIG. 4 is a block diagram of an example computer 400, i.e. a spoke computer 106 or a hub computer 108, as descried in FIG. 1, for implementing the system and method for facilitating a promotional event. The example computer 400 is intended to represent various forms of digital computers, including laptops, desktops, handheld computers, tablet computers, servers, and other similar types of computing devices. Computer 400 includes a processor 402, memory 404, a storage device 406, and a communication port 408, operably connected by an interface 410 via a bus 412.

Storage device 406 can store system 200 including invitation logic 202, authentication logic 204, synchronization logic 206, status logic 208, video logic 210, and communication logic 212.

Processor 402 processes instructions, via memory 404, for execution within computer 400, invitation logic 202, authentication logic 204, synchronization logic 206, status logic 208, video logic 210, and communication logic 212 stored on storage device 406. In an example embodiment, multiple processors along with multiple memories may be used.

Memory 404 may be volatile memory or non-volatile memory. Memory 404 may be a computer-readable medium, such as a magnetic disk or optical disk. Storage device 406 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 404 or storage device 406. The computer program product may contain system 200 including invitation logic 202, authentication logic 204, synchronization logic 206, status logic 208, video logic 210, and communication logic 212.

Computer 400 can be coupled to one or more input and output devices such as a display 414, a printer 416, a scanner 418, and a mouse 420.

While example systems, methods, and so on, have been illustrated by describing examples, and while the examples have been described in considerable detail, it is not the intention to restrict or in any way limit the scope of the appended claims to such detail. It is, of course, not possible to describe every conceivable combination of components or methodologies for purposes of describing the systems, methods, and so on, described herein. Additional advantages and modifications will readily appear to those skilled in the art. Therefore, the invention is not limited to the specific details, and illustrative examples shown or described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

Some portions of the detailed descriptions are presented in terms of algorithms and symbolic representations of operations on data bits within a memory. These algorithmic descriptions and representations are the means used by those skilled in the art to convey the substance of their work to others. An algorithm is here, and generally, conceived to be a sequence of operations that produce a result. The operations may include physical manipulations of physical quantities. Usually, though not necessarily, the physical quantities take the form of electrical or magnetic signals capable of being stored, transferred, combined, compared, and otherwise manipulated in a logic and the like.

1. A method for facilitating a healthcare promotional event, the method comprising the steps of:
a computer transmitting data representative of an invitation for a healthcare professional, selected from a plurality of healthcare professionals, to participate in a healthcare promotional event, selected from a plurality of predefined healthcare promotional events, wherein participating in a healthcare promotional event comprises watching a pre-recorded healthcare promotional video and participating in a live communication with a healthcare promotional event host;
a computer receiving confirmation of the healthcare professional's intent to participate in the healthcare promotional event;
a computer launching the healthcare promotional event by initiating playback of the pre-recorded healthcare promotional video responsive to the computer receiving notification that the healthcare professional has received a communication comprising pre-defined information associated with the pre-recorded healthcare promotional video; and
a computer establishing live communication with the healthcare promotional event host responsive to the computer receiving notification that the healthcare professional has completed watching the pre-recorded healthcare promotional video.

2. The method of claim 1, further comprising the steps of:
a computer receiving authentication credentials;
a computer filtering the plurality of healthcare professionals based on the authentication credentials; and
a computer filtering the plurality of predefined healthcare promotional events based on the authentication credentials.

3. The method of claim 1, further comprising the steps of:
a computer receiving notification indicative of whether the healthcare professional participated in the healthcare promotional event; and
a computer storing data indicative of whether the healthcare professional participated in the healthcare promotional event.

4. The method of claim 3, further comprising the step of a computer generating a report based on the stored data.

5. The method of claim 1, further comprising the steps of:
a computer receiving notification that a second healthcare professional, for which the computer did not transmit data representative of an invitation to participate in the healthcare promotional event, did participate in the healthcare promotional event; and
the computer storing data indicating that that second healthcare professional did participate in the healthcare promotional event.

6. The method of claim 5, further comprising the step of the computer storing data representative of the second healthcare professional responsive to determining that the second healthcare professional is not one of the plurality of healthcare professionals.

7. The method of claim 1, wherein the step of the computer establishing live communication with the healthcare promotional event host comprises the computer establishing a video conference session with the healthcare promotional event host.

8. A system for facilitating a healthcare promotional event, the system comprising:
a hub computer configured to host a live video conference with a healthcare key opinion leader associated with a healthcare promotional event; and
a spoke computer comprising at least one processor, at least one computer-readable tangible storage device, and program instructions stored on the at least one storage device for execution by the at least one processor, the program instructions comprising:
first program instructions configured to associate a healthcare professional with the healthcare promotional event;
second program instructions configured to initiate playback of a healthcare promotional video associated with the healthcare promotional event;
third program instructions configured to establish a connection, via the hub computer, to the live video conference with the healthcare key opinion leader; and
fourth program instructions configured to certify that the healthcare professional associated with the healthcare promotion event participated in the healthcare promotional event.

9. The system of claim 8, wherein the fourth program instructions are further configured to:
receive a first confirmation that the healthcare professional received information associated with the healthcare promotional video;
receive a second confirmation that the healthcare professional completed watching the healthcare promotional video; and
store data indicative of the first confirmation and the second confirmation.

10. The system of claim 8, further comprising a second spoke computer comprising at least one processor, at least one computer-readable tangible storage device, and program instructions stored on the at least one storage device for execution by the at least one processor, the program instructions comprising:
fifth program instructions configured to associate an additional healthcare professional, geographically separated from the healthcare professional, with the healthcare promotional event;
sixth program instructions configured to initiate playback of the healthcare promotional video associated with the healthcare promotional event;
seventh program instructions configured to establish a second connection, via the hub computer, to the live video conference with the healthcare key opinion leader; and
eighth program instructions configured to certify that the additional healthcare professional associated with the healthcare promotion event participated in the healthcare promotional event.

11. The system of claim 8, further comprising a storage computer configured to store data representative of healthcare professionals, data representative of predefined healthcare promotional events, and healthcare promotional videos, wherein the program instructions further comprise fifth program instructions configured to synchronize, with the storage computer, data representative of healthcare professionals, data representative of predefined healthcare promotional events, and healthcare promotional videos.

12. The system of claim 11, further comprising sixth program instructions configured to synchronize, with the storage computer, data representative of previously defined associations between healthcare professionals and promotional events.

13. The system of claim 12, further comprising seventh program instructions configured to receive authentication credentials, wherein the sixth program instructions are configured to synchronize, with the storage computer, data representative of previously defined associations between healthcare professionals and promotional events based on the received authentication credentials.

14. The system of claim 8, wherein the spoke computer is a portable tablet computer.

15. A computer program product for facilitating a virtual promotional event, the computer program product comprising:
at least one computer-readable tangible storage device and program instructions stored on the at least one storage device, the program instructions comprising:
first program instructions configured to transmit data representative of an invitation for an invitee, selected from a predefined dataset, to participate in a virtual promotional event, selected from a plurality of predefined virtual promotional events, wherein participating in a virtual promotional event comprises watching a pre-recorded promotional video and participating in a live communication with a virtual promotional event host;
second program instructions configured to receive confirmation of the invitee's intent to participate in the virtual promotional event and to change a status of an invitee from an invited status to a confirmed status, responsive to receiving the confirmation;
third program instructions configured to receive notification that the confirmed invitee has received a communication comprising pre-defined information associated with the promotional video, and in response, to launch the virtual promotional event by initiating playback of the pre-recorded promotional video; and
fourth program instructions configured to receive notification that the confirmed invitee has completed watching the pre-recorded promotional video, and in response, to establish communication with the virtual promotional event host.

16. The computer program product of claim 15, the program instructions further comprising:
sixth program instructions configured to receive authentication credentials;
seventh program instructions configured to filter the dataset based on the authentication credentials; and
eighth program instructions configured to filter the plurality of predefined virtual promotional events based on the authentication credentials.

17. The computer program product of claim 15, the program instructions further comprising:
sixth program instructions configured to receive notification indicative of whether the confirmed invitee participated in the virtual promotional event; and
seventh program instructions configured to store data indicating that that non-invited guest did participate in the virtual promotional event.

18. The computer program product of claim 15, the program instructions further comprising:
sixth program instructions configured to receive a notification that a non-invited guest, for which data representative of an invitation to participate in the virtual promotional event was not transmitted, did participate in the virtual promotional event; and
seventh program instructions configured to change a status of the non-invited guest to a status indicating that the non-invited guest did participate in the virtual promotional event.

19. The computer program product of claim 18, the program instructions further comprising eighth program instructions configured to store data representative of the non-invited guest responsive to determining that the non-invited guest is not included in the dataset.

20. The computer program product of claim 15, wherein the fifth program instructions configured to establish communication with the virtual promotional event host, is further configured to establish a video conference session with the virtual promotional event host.
